(19) United States
(12) Patent Application Publication
(10) Pub. No.: US 2017/0216188 A1
(43) Pub. Date: Aug. 3, 2017

(54) USE OF A COMBINATION OF POLYURETHANE-2/POLYMETHYL METHACRYLATES AND POLYVINYLPYRROLIDONE

(71) Applicant: Henkel AG & Co. KGaA, Duesseldorf (DE)

(72) Inventors: Maria Catalina Bermudez Agudelo, Darmstadt (DE); Julia Bibiane Lange, Bad Bramstedt (DE); Cyrielle Martinez, Hamburg (DE); Bernd Richters, Hamburg (DE)

(73) Assignee: Henkel AG & Co. KGaA, Duesseldorf (DE)

(21) Appl. No.: 15/492,322

(22) Filed: Apr. 20, 2017

Related U.S. Application Data
(63) Continuation of application No. PCT/EP2015/070426, filed on Sep. 8, 2015.

(30) Foreign Application Priority Data
Oct. 21, 2014 (DE) 102014221366.5

(51) Int. Cl. A61K 8/87; A61K 8/81; A61K 8/04; A61Q 5/06

(52) U.S. Cl. CPC A61K 8/87; A61Q 5/06; A61K 8/8176; A61K 8/04; A61K 2800/594

(57) ABSTRACT

The present invention relates to a cosmetic composition for fixing hair or for temporarily shaping keratinous fibers, in particular, human hair, with which composition an excellent resistance to humidity is achieved, wherein the composition includes a combination of polyvinylpyrrolidone (PVP) and polyurethene-polyacrylate hybrid polymer.

### FIELD OF THE INVENTION

The present invention generally relates to a cosmetic composition for fixing hair or for temporarily shaping keratinous fibers, in particular, human hair, with which composition an improved resistance to humidity is achieved, wherein the composition includes a combination of polyvinylpyrrolidone (PVP) and a specific film-forming polymer.

### BACKGROUND OF THE INVENTION

Temporary shaping of hairstyles for a longer period of time, up to several days, generally requires the use of fixing agents. Therefore, hair treatment agents that are used to temporarily give shape to the hair play an important role. Corresponding agents for temporary shaping typically include synthetic polymers and/or waxes as fixing agents. Agents for supporting the temporary reshaping of hair may be prepared, for example, as a hair spray, hair wax, hair gel, or hair mousse.

The most important property of an agent for temporarily shaping hair-hereinafter also called a styling agent-is giving the strongest possible hold to the treated fibers in the newly-modeled form, i.e., a form imparted to the hair. Other topics of discussion are strong hairstyle holding or a high degree of holding of the styling agent. The hairstyle hold is determined essentially by the type and amount of the fixing agent used, wherein, however, the other components of the styling agent may also have an effect.

In addition to a high degree of hold, styling agents need to fulfill a whole range of other requirements. These can be broadly divided into: properties on the hair; properties of the respective formulation, e.g., properties of the foam, gel, or sprayed aerosol; and properties that affect the handling of the styling agent, wherein the properties on the hair are of special importance. These include, in particular, resistance to humidity, low tackiness, and a balanced conditioning effect. Furthermore, a styling agent should be universally applicable to all hair types, as much as possible, and should be mild on the hair and skin. To meet the different requirements, a large number of synthetic polymers that are used in styling agents have already been developed as fixing agents. The polymers can be divided into cationic, anionic, nonionic, and amphoteric fixing polymers. Ideally, when applied to the hair, the polymers produce a polymer film that, on one hand, gives very strong hold to the hairstyle but, on the other hand, is sufficiently flexible to not break under stress. If the polymer film is too brittle, so-called film plaques can develop (i.e., residues that are shed with movement of the hair and give the impression that the user of the respective styling agent has dandruff). Similar problems arise when waxes are put to use as a fixing agent in the styling agent. If the styling agent entails a gel or paste, the polymers should also possess thickening properties.

Known nonionic polymers that are put to use in hair-fixing products are polyvinylpyrrolidones (PVPs). These are homopolymers of vinylpyrrolidone. Vinylpyrrolidone homopolymers are marketed, for example, under the name Luviskol® (BASF) and are commonly used as film formers and/or thickening agents. In particular, the product series Luviskol® K (BASF), in which vinylpyrrolidone homopolymers are offered in a variety of molecular weights, is available for use in cosmetic compositions and styling products as a film former or thickening agent.

Additional known polymers that have also been put to use as ingredients in cosmetic compositions, e.g., styling agents, are hybrid polymers of polyurethane and polyacrylate. Examples of such copolymers, which are also known as interpenetrating polymer networks, are hybrid polymers made of polyurethane and polymethyl methacrylate. For cosmetic uses, such polymers are commercially available under the name Hybridur® from the company Air Products and Chemicals, Inc., USA. Hybridur® 875 Polymer Dispersion is an aqueous polyurethane-polyacrylate dispersion with the INCI designation Polyurethane-2 and Polymethyl Methacrylate. Polyurethane-2 is a copolymer made of the monomers of hexylene glycol, neopentyl glycol, adipic acid, methylene diphenyl diisocyanate, and dimethylpropionic acid. Hybridur®875 Polymer Dispersion is described by the manufacturer as ideal for obtaining high resistance to humidity in haircare products.

EP 2286908 A2 and EP 2455157 A1 describe Hybridur® 875 as a component of hair gels. WO 2014012918 A2 describes aqueous dispersions of polyurethane/polyacrylate hybrid polymers as a component of styling agents.

This was achieved according to the present invention by a combination of two certain nonionic polymers.

### BRIEF SUMMARY OF THE INVENTION

A cosmetic composition for temporarily shaping keratinous fibers, which includes:

a) a vinylpyrrolidone homopolymer, and

b) a polyurethane-polyacrylate hybrid polymer.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has, within the scope of the present invention, been surprisingly and unexpectedly discovered and confirmed that the combination of two components, known per se, which have already been put to use in styling products, makes it possible to obtain styling products with improved resistance to humidity. Other commonly required properties of styling products, such as long-term hold, rigidity, and low tackiness remained unchanged. Such a favorable combination of properties was not expected, even with knowledge of the individual components used, and was surprising. It has been shown experimentally that the combination of the two components yields a synergistic effect with regard to the resistance to humidity, which has manifested itself in the high-humidity curl retention (HHRC) test.

According to the present invention, the term “keratinous fibers” encompasses fur, wool, and feathers, but especially human hair.

The essential components of the cosmetic composition according to the present invention are the vinylpyrrolidone homopolymer (a) and the polyurethane-polyacrylate hybrid polymer.

The agent according to the present invention compulsorily includes a vinylpyrrolidone homopolymer as the component (a). It is preferred according to the present invention to select the vinylpyrrolidone homopolymers from vinylpyrrolidone homopolymers having a K value (1-wt % solution of PVP, Brookfield at 23° C.) in water of 20 to 100. More preferable is a K value of 80 to 100, more preferably approximately 90. The K value, also known as intrinsic viscosity, is a parameter for characterizing polymers, which is easily determined from the relative viscosity by means of viscosity measurements taken from polymer solutions.

Preferred vinylpyrrolidone homopolymers are available under the tradenames Luviskol® K 30, Luviskol® K 80, Luviskol® K 85, and Luviskol® K 90, each from the company BASF SE. Luviskol® K 90 is the most preferred, according to the present invention. Luviskol K90 is a 20% aqueous, colorless to pale yellow solution of polyvinylpyrrolidone. The product has a K value of 90.0 to 98.0 (1% (m/V) in water), a solids content of 19.0 to 21.0 wt %, and a pH value of 7.0 to 9.0 (10 wt % solids content in water).

The component (b) used according to the present invention is a hybrid polymer made of polyurethane and polyacrylate. Examples of such copolymers, which are also known as interpenetrating polymer networks, are hybrid polymers made of polyurethane and polymethyl methacrylate (see, for example, S.C. Kim et al., Macromolecules, 1976, 9(2), S. 258-263). Preferably, the polyurethane-polyacrylate hybrid polymer (b) is used as an aqueous dispersion.

Further preferably, the polyurethane of the polyurethane-polyacrylate hybrid polymer (b) is a copolymer of the monomers of hexylene glycol, neopentyl glycol, adipic acid, methylene diphenyl diisocyanate, and dimethylpropionic acid (INCI: Polyurethane-2).

Further preferably, the polyacrylate of the polyurethane-polyacrylate hybrid polymer (b) is a polymethyl methacrylate. Most preferably, the polyurethane-polyacrylate hybrid polymer (b) is one with the INCI designation Polyurethane-2 and Polymethyl Methacrylate. This is available under the tradename Hybridur® 875 Polymer Dispersion (Air Products and Chemicals, Inc., USA) as an approximately 40 wt % dispersion (typical properties: anionic, pH approximately 7.5 to 9.0, viscosity Brookfield cPs approximately 50 to 100, particle size approximately 75 to 80 nm). It is especially preferable according to the present invention to use a polyurethane-polyacrylate hybrid polymer (b) with the INCI designation Polyurethane-2 and Polymethyl Methacrylate, in particular, Hybridur® 875 (Air Products and Chemicals Inc.), in combination with a vinylpyrrolidone homopolymer having a K value (1-wt % solution of PVP, Brookfield, at 23° C.) in water of 20 to 100, more preferably 80 to 100, still more preferably approximately 90, in particular, Luviskol® K90−20%, BASF. With this combination, according to the present invention, an especially favorable resistance to humidity was obtained in the hair styling agent. Further required properties of styling products, such as long-term hold, rigidity, and low tackiness are also achieved in particular with this combination.

The cosmetic composition of the present invention includes the vinylpyrrolidone homopolymer (a) and the polyurethane-polyacrylate hybrid polymer (b) in quantities that are common and suitable for styling agents, which can be adapted to the specific use and manufacturing.

Relative to the total weight of the agent according to the present invention, the vinylpyrrolidone homopolymer (a) is included, for example, in a total amount of 0.01 to 5.0 wt %, preferably 0.1 wt % to 3.0 wt %, more preferably 0.2 to 2.5 wt %, especially preferably 0.3 wt % to 2.0 wt %, very especially preferably 0.4 to 1.4 wt %, and especially preferably 0.8 to 1.2 wt % in the agent according to the present invention, each case being set forth as the solids content of active substances in the cosmetic composition.

The cosmetic composition according to the present invention includes the polyurethane-polyacrylate hybrid polymer (b)—relative to the total weight of the cosmetic composition—for example, in an amount of 0.05 to 5.0 wt %, preferably 0.1 to 4.0 wt %, further preferably 0.2 to 3.0 wt %, also preferably 0.4 to 2.0 wt %, also preferably 0.5 to 1.5 wt %, and especially preferably 0.8 to 1.2 wt %, each case being set forth as the solids content of active substances in the cosmetic composition.

Preferably, the cosmetic composition of the present invention includes one or more other components acting as thickening agents or gelling agents, which are different from the components (a) and (b) and also support the film formation. Examples include cationic, anionic, nonionic, or amphoteric polymers. The proportion by weight of these other components to the total weight of the cosmetic composition is, for example, 0.02 to 3 wt %, preferably 0.05 to 1.5 wt %, and still more preferably 0.2 to 0.8 wt %.

Examples include Acrylamide/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/t-Butylacrylamide Copolymer, Acrylates Copolymer, Acrylates/Cl-2 Succinates/Hydroxyacrylates Copolymer,

Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetoneacrylamide Copolymer, Ammonium VA/Acrylates Copolymer, AMPD-Acrylates/Diacetoneacrylamide Copolymer, AMP-Acrylates/ Allyl Methacrylate Copolymer, AMP-Acrylates/Cl-18 Alkyl Acrylates/Cl-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Com Starch/Acrylamide/ Sodium Acrylate Copolymer, Diethylene Glycolamine/Epichlorohydrin/Piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-Hydroxypropyl Dimethicone Acrylate Copolymer, Isopropyl Ester of PVM/MA Copolymer, Lauryl Acrylate Crosspolymer, Lauryl Methacrylate/Glycol Dimethacrylate Crosspolymer, MEA-Sulfite, Methacrylic Acid/Sodium Acrylamidomethyl Propane Sulfonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone/Acrylates Copolymer, PEG-8/SMDI Copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-51/SMDI Copolymer, PPG-10 Sorbitol, PVM/MA Copolymer, PVP, PVP/VA/ltaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylsiloxysilylcarbamoyl Pullulan, VA/Crotonates Copolymer, VA/C roto nate s/Methacryloxyb enzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate, and Styrene/VP Copolymer.

UTILITY PATENT APPLICATION FILED EFS-WEB

ATTORNEY DOCKET NO. PT032813

Examples of nonionic polymers are:
1. Vinylpyrrolidone/vinyl ester copolymers, such as are marketed under the trademark Luviskol (BASF). Luviskol VA 64 and Luviskol VA 73—each vinylpyrrolidone/vinyl acetate copolymers—are preferred nonionic polymers.
2. Cellulose ethers, such as hydroxypropyl cellulose, hydroxyethyl cellulose, and methylhydroxypropyl cellulose, such as are marketed, for example, under the trademarks Culminal and Benecel (AQUALON).
3. Shellac.
4. Siloxanes. These siloxanes may be both water-soluble and water-insoluble. Both volatile and non-volatile siloxanes are suitable, wherein non-volatile siloxanes are understood to be those compounds that have a boiling point at atmospheric pressure above 200° C. Preferred siloxanes are polydialkyl siloxanes, such as for example polydimethyl siloxane, polyalkylaryl siloxanes, such as for example polyphenylmethyl siloxane, ethoxylated polydialkyl siloxanes as well as polydialkyl siloxanes including amine and/or hydroxy groups.
5. Glyco sidically-substituted silicones.

Preferably, the addition component acting as a gelling agent is a homopolyacrylic acid (INCI: Carbomer), which is commercially available under the name Carbopol® in a variety of different versions. The carbomer is preferably included in a proportion of 0.02 to 3.0 wt %, preferably 0.05 to 1.5 wt %, and still more preferably 0.2 to 0.8 wt %, relative to the total weight of the cosmetic composition.

The cosmetic composition according to the present invention may include other common ingredients of styling products. Other suitable auxiliary substances and additives include, in particular, additional conditioners.

As a conditioner of another compound class, the agent may include, for example, at least one protein hydrolyzate and/or a derivative thereof. Protein hydrolyzates are product mixtures that are obtained by acidically, basically, or enzymatically catalyzed breakdown of proteins. The term “protein hydrolyzates” is also understood according to the present invention to mean total hydrolyzates as well as individual amino acids and derivatives thereof, and mixtures of different amino acids. The molecular weight of the protein hydrolyzates that can be used according to the present invention lies between 75 (the molar weight of glycine) and 200,000; preferably, the molecular weight is 75 to 50,000, very especially preferably 75 to 20,000 Daltons.

As a conditioner, the agent according to the present invention may furthermore include at least one vitamin, provitamin, vitamin precursor, and/or derivative thereof. Those vitamins, provitamins, and vitamin precursors that are usually assigned to the groups A, B, C, E, F, and H are preferred according to the present invention.

Similar to the addition of glycerol and/or propylene glycol, the addition of panthenol enhances the flexibility of the polymer film that is formed when the agent according to the present invention is used.

As a conditioner, the agents according to the present invention may furthermore include at least one plant extract, but also mono- or oligosaccharides and/or lipids.

Oil components are also suitable as conditioners. Natural and synthetic cosmetic oil components include, for example, plant oils, liquid paraffin oils, isoparaffin oils, and synthetic hydrocarbons, as well as di-n-alkyl ethers having a total of 12 to 36 C atoms, especially 12 to 24 C atoms. Preferred cosmetic agents according to the present invention include at least one oil component, preferably at least one oil component from the group consisting of silicone oils. The group of silicone oils comprises, in particular, dimethicones—which also include cyclomethicones—and amino-functional silicones and dimethiconols. Dimethicones can be both linear and branched, as well as cyclic or cyclic and branched. Suitable silicone oils or silicone gums are, in particular, dialkyl- and alkylarylsiloxanes, such as, for example, dimethylpolysiloxane and methylphenylsiloxane, as well as alkoxylated, quaternized, or also anionic derivatives thereof. Cyclic and linear polydialkyl siloxanes, alkoxylated and/or aminated derivatives thereof, dihydroxypolydimethyl siloxanes, and polyphenylalkyl siloxanes are preferred.

Other preferred nourishing oil components are ester oils, i.e., esters of C₆-C₃₀ fatty acids with C₂-C₃₀ fatty alcohols, preferably monoesters of fatty acids with alcohols having 2 to 24 C atoms, such as, for example, isopropyl myristate (Rilanit® IPM), isononanoic acid C₁₆₋₁₈ alkyl esters (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), and oleic acid decyl ester (Cetiol® V).

Also suitable as conditioners are dicarboxylic acid esters, symmetric, asymmetric, or cyclic esters of carbonic acid with fatty alcohols, tri-fatty acid esters of saturated and/or unsaturated, linear and/or branched fatty acids with glycerol, or fatty acid partial glycerides, which are understood to be monoglycerides, diglycerides, and technical mixtures thereof.

Furthermore, the composition according to the present invention preferably includes emulsifiers or surface-active agents. PEG derivatives of hydrogenated castor oil, available under the designation PEG Hydrogenated Castor Oil, are preferred; examples include PEG-30 Hydrogenated Castor Oil, PEG-33 Hydrogenated Castor Oil, PEG-35 Hydrogenated Castor Oil, PEG-36 Hydrogenated Castor Oil, or PEG-40 Hydrogenated Castor Oil. The use of PEG-40 Hydrogenated Castor Oil is preferred according to the present invention. These are preferably included in an amount of 0.05 to 1.5 wt %, more preferably 0.1 to 1.0 wt %, also preferably 0.2 to 0.8 wt % or 0.3 to 0.6 wt %.

To adjust the pH, the cosmetic composition according to the present invention may furthermore include neutralizers or pH adjusting agents. Examples of neutralizers that are used in styling products include primary amino alcohols. An example of such a neutralizer that can preferably be used according to the present invention is Aminomethyl Propanol (INCI), which is commercially available, for example, under the name AMP-ULTRA® PC. AMP-ULTRA® PC 2000 is preferred according to the present invention, and includes 5% water.

The agents according to the present invention include the ingredients or active ingredients in a cosmetically acceptable carrier.

Preferred cosmetically acceptable carriers are aqueous, alcoholic, or aqueous-alcoholic media with preferably at least 10 wt % water, calculated relative to the total weight of the agent.

Especially preferably, the cosmetic carrier according to the present invention includes water, in particular, in such an amount that the cosmetic agent—as calculated relative to the total weight of the agent—includes at least 10 wt %, in particular, at least 20.0 wt %, further preferably at least 40 wt %, also preferably at least 70 wt % water.

As alcohols, in particular, the lower alcohols commonly used for cosmetic purposes, with 1 to 4 carbon atoms, such as ethanol and isopropanol, maybe included.

Examples of water-soluble solvents as a cosolvent include glycerol and/or ethylene glycol and/or 1,2-propylene glycol in an amount of 0 to 30 wt % relative to the entire agent.

The cosmetic composition of the present invention may be manufactured in the forms that are usual for temporarily shaping hair, e.g., hair gel, hair spray, hair mousse, or hair wax.

The present invention also relates to the use of cosmetic compositions according to the present invention to temporarily shape keratinous fibers, in particular, human hair, and a method for temporarily shaping keratinous fibers, in particular, human hair, with which the cosmetic composition according to the present invention is applied to keratinous fibers.

Tabular overview

The compositions of some preferred cosmetic agents can be found in the following tables (all amounts given as solids content and in wt % relative to the total weight of the cosmetic agent. unless otherwise indicated).

| | Formula | Formula | Formula | Formula | Formula |
|---|---|---|---|---|---|
| | 1 | 2 | 3 | 4 | 5 |
| Vinyl- | 0.1 to 3.0 | 0.2 to 2.5 | 0.3 to 2.0 | 0.4 to 1.4 | 0.8 to 1.2 |
| pyrrolidone | | | | | |
| homo- | | | | | |
| polymer (a) | | | | | |
| Polyurethane- | 0.1 to 4.0 | 0.2 to 3.0 | 0.4 to 2.0 | 0.5 to 1.5 | 0.8 to 1.2 |
| polyacrylate | | | | | |
| hybrid polymer | | | | | |
| (b) | | | | | |
| Misc | up to 100 | up to 100 | up to 100 | up to 100 | up to 100 |

| | Formula | Formula | Formula | Formula | Formula |
|---|---|---|---|---|---|
| | 1a | 2a | 3a | 4a | 5a |
| Vinyl- | 0.1 to 3.0 | 0.2 to 2.5 | 0.3 to 2.0 | 0.4 to 1.4 | 0.8 to 1.2 |
| pyrrolidone | | | | | |
| homopolymer | | | | | |
| (a): | | | | | |
| Luviskol ® | | | | | |
| K90-20% | | | | | |
| Polyurethane- | 0.1 to 4.0 | 0.2 to 3.0 | 0.4 to 2.0 | 0.5 to 1.5 | 0.8 to 1.2 |
| polyacrylate | | | | | |
| hybrid | | | | | |
| polymer (b): | | | | | |
| INCI: | | | | | |
| Polyurethane-2 | | | | | |
| and Polymethyl | | | | | |
| Methacrylate | | | | | |
| Misc | up to 100 | up to 100 | up to 100 | up to 100 | up to 100 |

| | Formula | Formula | Formula | Formula | Formula |
|---|---|---|---|---|---|
| | 11 | 12 | 13 | 14 | 15 |
| Vinyl- | 0.1 to 3.0 | 0.2 to 2.5 | 0.3 to 2.0 | 0.4 to 1.4 | 0.8 to 1.2 |
| pyrrolidone | | | | | |
| homo- | | | | | |
| polymer | | | | | |
| (a) | | | | | |
| Poly- | 0.1 to 4.0 | 0.2 to 3.0 | 0.4 to 2.0 | 0.5 to 1.5 | 0.8 to 1.2 |
| urethane- | | | | | |
| poly- | | | | | |
| acrylate | | | | | |
| hybrid | | | | | |
| polymer | | | | | |
| (b) | | | | | |
| Carbomer | 0.02 to 3.0 | 0.05 to 2.0 | 0.1 to 1.5 | 0.2 to 1.0 | 0.4 to 0.8 |
| Misc | up to 100 | up to 100 | up to 100 | up to 100 | up to 100 |

| | Formula 11a | Formula 12a | Formula 13a | Formula 14a | Formula 15a |
|---|---|---|---|---|---|
| Vinylpyrrolidone | 0.1 to 3.0 | 0.2 to 2.5 | 0.3 to 2.0 | 0.4 to 1.4 | 0.8 to 1.2 |
| homopolymer | | | | | |
| (a): Luviskol ® | | | | | |
| K90-20% | | | | | |
| Polyurethane- | 0.1 to 4.0 | 0.2 to 3.0 | 0.4 to 2.0 | 0.5 to 1.5 | 0.8 to 1.2 |
| polyacrylate hybrid | | | | | |
| polymer | | | | | |
| (b): INCI: | | | | | |
| Polyurethane-2 | | | | | |
| and Polymethyl | | | | | |
| Methacrylate | | | | | |
| Carbomer | 0.02 to 3.0 | 0.05 to 2.0 | 0.1 to 1.5 | 0.2 to 1.0 | 0.4 to 0.8 |
| Misc | up to 100 | up to 100 | up to 100 | up to 100 | up to 100 |

“Misc” is understood according to the present invention to mean a cosmetic carrier, in particular, water and optionally other usual components of styling products.

### EXAMPLES

The following styling agent was produced:

| Components/ | | | |
|---|---|---|---|
| raw materials | wt % | INCI designation | Manufacturer |
| Water, | 92.500 | | |
| demineralized | | | |
| Hybridur ® 875 | 2.5 | Polyurethane-2 | Air Products and |
| Polymer | | and Polymethyl | Chemicals, Inc. |
| Dispersion | | Methacrylate | |
| Luviskol ® | 5.00 | Vinylpyrrolidone | BASF |
| K90-20% | | homopolymer | |
| Total | 100 | | |

The amounts set forth in the table are in wt % of the respective raw material, relative to the entire composition.

The solids content of the Polyurethane-2 and Polymethyl Methacrylate and of the vinylpyrrolidone homopolymer thus amounted to 1 wt %.

The resulting styling agent showed an exceptionally favorable resistance to humidity in the high-humidity curl retention (HHCR) test.

After 6 hours, values of 31.8 to 38.1 were obtained in the HHCR test (mean: 34.95% when determined on 10 curly hair strands).

Other required properties, such as rigidity, combability, and long-term hold, were also obtained.

For comparison, HHRC values for Hybridur® 875 in water with a solids content of 2 wt % and for Luviskol® K90 20% in water with a solids content of 2 wt % were determined at the same time.

Results:

Hybridur® 875: Values of 23 to 30%, mean: 26.9 when determined on 5 curly hair strands.

Luviskol® K90: Values of 26.5 to 28.5%, mean: 27.7 when determined on 5 curly hair strands.

Thus, there was shown to be a synergistic effect on the resistance to humidity for the combination “Polyurethane-2 and Polymethyl Methacrylate” and “Vinylpyrrolidone Homopolymer”.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic composition for temporarily shaping keratinous fibers, comprising:
a. a vinylpyrrolidone homopolymer, and
b. a polyurethane-polyacrylate hybrid polymer.

2. The cosmetic composition according to claim 1, wherein the polyurethane-polyacrylate hybrid polymer is in the form of an aqueous dispersion.

3. The cosmetic composition according to claim 1, wherein the polyurethane is a copolymer of the monomers of hexylene glycol, neopentyl glycol, adipic acid, methylene diphenyl diisocyanate, and dimethylpropionic acid.

4. The cosmetic composition according to claim 1, wherein the polyacrylate is polymethyl methacrylate.

5. The cosmetic composition according to claim 1, wherein the vinylpyrrolidone homopolymer (a) has a K value (1-wt % solution of PVP, Brookfield at 23° C.) in water of 20 to 100.

6. The cosmetic composition according to claim 1, wherein the vinylpyrrolidone homopolymer (a) has a K value (1-wt % solution of PVP, Brookfield at 23° C.) in water of 80 to 100.

7. The cosmetic composition according to claim 1, wherein the polyurethane-polyacrylate hybrid polymer has the INCI designation Polyurethane-2 and Polymethyl Methacrylate.

8. The cosmetic composition according to claim 1, comprising, relative to the total weight of the cosmetic composition 0.2 to 3.0 wt % the vinylpyrrolidone homopolymer (a), and 0.2 to 2.0 wt % the polyurethane-polyacrylate hybrid polymer (b).

9. The cosmetic composition according to claim 1, comprising, relative to the total weight of the cosmetic composition, 0.4 to 2.0 wt % the vinylpyrrolidone homopolymer (a), and 0.5 to 1.5 wt % the polyurethane-polyacrylate hybrid polymer (b).

10. The cosmetic composition according to claim 1, comprising, relative to the total weight of the cosmetic composition, 0.05 to 1.5 wt % a carbomer.

11. The cosmetic composition according to claim 1, wherein the cosmetic composition is in the form of a hair gel, hair spray, hair mousse, or hair wax.
